(19) United States
(12) Patent Application Publication
(10) Pub. No.: US 2003/0220626 A1
(43) Pub. Date: Nov. 27, 2003

(54) Loopless absorbent article

(76) Inventor: Hamzeh Karami, Brewster, MA (US)

Correspondence Address: Neal L. Rosenberg, Esq., AMSTER, ROTHSTEIN & EBENSTEIN, 90 Park Avenue, New York, NY, 10016, US

(21) Appl. No.: 10/431,200

(22) Filed: May 7, 2003

Related U.S. Application Data
(63) Continuation-in-part of application No. 10/134,346, filed on Apr. 26, 2002.
(63) Continuation-in-part of application No. 09/797,335, filed on Mar. 1, 2001, now Pat. No. 6,520,826.
(63) Continuation-in-part of application No. 09/544,092, filed on Apr. 6, 2000, now abandoned.
(63) Continuation-in-part of application No. 09/376,282, filed on Aug. 18, 1999.

(51) Int. Cl. A61F 13/15

(52) U.S. Cl. 604/396

(57) ABSTRACT

A disposable absorbent article includes a spunbond nonwoven forming at least one of an article backsheet and article frontsheet and defining an exposed outer surface thereof containing a plurality of interstices, and a minihook fastener element forming a plurality of pins defining an exposed outer surface thereof. The pins of the fastener element and the interstices of the nonwoven are configured and dimensioned to provide, when the exposed outer surfaces of the fastener element and the nonwoven are mutually engaged, a peel strength of at least 60 grams per 2 inches and a shear strength of at least 1300 grams per 2 inches.

### CROSS-REFERENCE TO RELATED APPLICATIONS

This is a continuation-in-part of U.S. application Ser. No. 10/134,346, filed Apr. 26, 2002, itself a continuation-in-part of U.S. application Ser. No. 09/797,335, filed Mar. 1, 2001, itself a continuation-in-part of U.S. application Ser. No. 09/544,092, filed Apr. 6, 2000, itself a continuation-in-part of U.S. application Ser. No. 09/376,282, filed Aug. 18, 1999.

### BACKGROUND OF THE INVENTION

The present invention relates to an absorbent article, and more particularly to an absorbent article having a fastener element which does not require a special loop-providing landing zone.

Since the advent of VELCRO fastener assemblies wherein a hook-type fastener element composed of a plurality of minihooks (very small hooks) engages a loop-type fastener element composed of a multitude of miniloops (very small loops), their use has become more or less ubiquitous in the field of disposable absorbent articles (such as diapers, T-shape briefs and preformed pull-ups) due to their convenience, reliability and inexpensiveness. A primary limitation on their widespread use has been the need for each minihook fastener element to have a corresponding miniloop fastener element to engage with—i.e., to serve as a loop-type “landing zone” for the minihook fastener element.

Thus, instead of an article or article portion containing a minihook fastener element capable of engaging anywhere along a facing surface of another article or article portion, it has been necessary for the two articles or article portions to be designed such that, during use, the minihook and miniloop fastener elements will be appropriately positioned opposite one another for engagement with one another. This disadvantage of the fastener assembly has been ameliorated to some degree by the use of minihooks fastener elements of relatively small longitudinal length (e.g., a fraction of an inch) adapted to engage miniloop fastener elements of relatively great longitudinal length (e.g., several inches) so that each minihook fastener element could be positioned as desired anywhere along the length of each miniloop fastener element. (The preference for a small minihook fastener element relative to a large miniloop fastener element, rather than vice versa, is founded on both the relative inexpensiveness and the relatively softer, less irritating nature of the latter). Thus, conventional woven or nonwoven articles have had affixed to the surface thereof along one axis (e.g., sewn thereon) a miniloop fastener element strip of greater longitudinal dimensions then the corresponding minihook fastener element, thereby to provide the user with freedom of choice as to where the minihook fastener element would be positioned along the miniloop fastener element strip. For example, see U.S. Pat. No. 5,383,872, U.S. Pat. No. 5,624,429; U.S. Pat. No. 5,527,305; and U.S. Patent Application Publication No. 2002/0038110 A1. In this manner the user is provided with a degree of flexibility or choice along one axis, albeit at additional material and manufacturing costs for the strip. It has even been proposed that one entire surface of an article be constituted of a miniloop fastener element in order to provide the user with total flexibility and choice (that is, the ability to position the minihook fastener element anywhere on the article surface), albeit at an even greater material and manufacturing cost for the surface. See, for example, U.S. Pat. No. 5,019,065.

Disposable absorbent articles of the types mentioned hereinabove have different requirements as to the peel strength and shear strength of the various engagements depending, for example, on whether the engagement is about a waist or elsewhere, whether its fastening power is to be supplemented by the power of additional and possibly different fastener systems, etc. Generally a peel strength of at least 60 grams per square inch and a shear strength of at least 1300 grams per square inch is preferred.

The need to provide a miniloop fastener element—whether as a landing zone, as a strip, or as an entire surface—has stifled the development of uses for the minihook fastener element in various situation where it might prove advantageous—for example, to provide size adaptability (especially to enable an absorbent article to accommodate both persons of normal waist size and persons of smaller than normal waist size), to enable employment in situations where the benefit to be obtained does not economically justify the added material and manufacturing costs of providing both minihook and miniloop fastener elements, etc.

Accordingly, an object of the present invention to provide a disposable absorbent article which, in one preferred embodiment, incorporates a fastener assembly consisting exclusively of a minihook fastener element without any miniloop fastener element beyond a conventional nonwoven material.

Another object to provide such an absorbent article wherein, in one preferred embodiment, the fastener assembly is composed exclusively of a minihook fastener element and a nonwoven (preferably a spunbond nonwoven and optimally an elastic composite thereof).

A further object is to provide such an absorbent article wherein, in one preferred embodiment, the fastener assembly provides a peel strength of at least 60 grams per square inch and a shear strength of at least 1300 grams per square inch.

It is also an object of the present invention to provide such an absorbent article wherein, in one preferred embodiment, the fastener assembly provides size adjustability or other features at the cost of only additional minihook fastener elements.

It is another object to provide such a disposable article wherein, in one preferred embodiment, the article is one of a diaper, a T-shaped brief, and a pull-up.

### SUMMARY OF THE INVENTION

It has now been found that the above and related objects of the present invention are obtained in a disposable absorbent article according to the present invention.

In a first preferred embodiment, the disposable absorbent article has a frontsheet and a backsheet and comprises a nonwoven defining an exposed outer surface of at least one of a frontsheet and a backsheet, the nonwoven containing a plurality of bonded fibers and a plurality of interstices defined thereby. The article further comprises a minihook fastener element forming a plurality of pins defining an exposed outer surface thereof. The pins of the fastener element and the fibers and the interstices of the nonwoven are configured and dimensioned to provide, when the exposed outer surfaces of the fastener element and the nonwoven are mutually engaged, a peel strength of at least 60 grams per square inch and a shear strength of at least 1300 grams per square inch.

In a second preferred embodiment, the article comprises a nonwoven defining an exposed outer surface of at least one of a frontsheet and a backsheet, the nonwoven containing a plurality of bonded fibers and a plurality of interstices defined thereby. The article further comprises a minihook fastener element. The fastener element has an average density of at least 1600 pins per square inch (PPSI) defining an exposed outer surface thereof, the pins being substantially circular and on average spaced less than about 300 microns apart at an exposed outer surface thereof, and includes a pin-bearing surface area of at least 0.5 inch². Preferably the pins are on average spaced less than about 275 microns apart at an exposed outer surface thereof, and the nonwoven has a basis weight of 13-50 gsm.

In a third preferred embodiment, the article comprises a nonwoven defining an exposed outer surface of at least one of a frontsheet and a backsheet, the nonwoven containing a plurality of bonded fibers and a plurality of interstices defined thereby. The nonwoven is a 13-50 gsm spunbond, the fibers have an average size of about 1.3-3.0 denier, and the average size of the interstices is at least 644 micron². The article further comprises a minihook fastener element forming a plurality of pins defining an exposed outer surface thereof, each pin having an average diameter at the base thereof of at least 300 (preferably 390) microns and at the free end thereof of at least 375 (preferably 427) microns. The pins of the fastener element enter into the interstices of the nonwoven when the exposed outer surfaces of the fastener elements and the nonwoven are mutually engaged. Preferably the fibers are differentially bonded, optimally differentially point bonded.

Preferably the nonwoven is the article backsheet or, in the case of a T-shaped brief, the article wings.

The absorbent article is preferably a diaper, T-shaped brief or pull-up. The diaper has a back portion, a front portion and a crotch portion connecting the back and front portions, one of the back and front portions including at least a laterally spaced pair of the fastener elements and the other of the back and front portions including the nonwoven. The T-shaped brief has a back portion, a front portion, a crotch portion connecting the back and front portions, and a pair of wings extending laterally from respective lateral edges of the back portion for partially overlapping connection about the front of the waist of a wearer, one of the wings having at least one of the fastener elements and the front portion including at least a first laterally spaced pair of the fastener elements adjacent a top thereof, and both of the wings including the nonwoven, whether elastic or non-elastic. The pull-up has a back portion, a front portion and a crotch portion connecting the back and front portions, the back and front portions being adapted to be secured together in an emergency situation exclusively by at least one of the fastener elements and the nonwoven.

In one embodiment of the T-shaped brief, the front portion includes a single auxiliary fastener element between the first pair of fastener elements and adjacent a top of the front portion. Alternatively, the front portion includes a second laterally spaced pair of the fastener elements adjacent a top thereof, the second pair being disposed between the first pair. Alternatively, the first laterally spaced pair of fastener elements is substantially spaced inwardly from the lateral edges of the front portion.

If desired, the lateral edges of the front portion adjacent a top thereof are secured in a turned-under orientation to the frontsheet of the front portion, the first laterally spaced pair of fastener elements being on the backsheet of the turned-under lateral edges. Alternatively, the first laterally spaced pair of fastener elements is disposed on the frontsheet of the front portion, and the front portion is disposed over at least one of said wings. Alternatively, the first laterally spaced pair of said fastener elements is disposed on the backsheet of the front portion, and the front portion is partially disposed under at least one of the wings.

In another embodiment of the T-shaped brief, the partially overlapped one of the wings has an auxiliary fastener element on the backsheet thereof adjacent the free end thereof, thereby to enable fastening of the free end to the frontsheet of the overlapping one of the wings.

In a further embodiment of the T-shaped brief, the partially overlapped one of the wings has an auxiliary fastener element on the backsheet thereof on an intermediate portion spaced from the free end thereof, thereby to enable fastening of the intermediate portion to the frontsheet of the overlapping one of the wings.

Preferably the wings are at least partially elastic, the nonwoven is a spunbond, and the nonwoven is at least the outer surface of the backsheet or a wing.

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the accompanying drawing wherein:

FIG. 1 is a top plan view (in a laid-out, fully stretched orientation on its backsheet) of a first embodiment of a disposable absorbent article according to the present invention, showing a T-shaped brief having three minihook fastener elements and a nonwoven;

FIG. 2 is a top plan view of a variant thereof;

FIGS. 3-6 are fragmentary top plan views of the front portion of additional variants thereof;

FIG. 7 is an isometric view of a disposable absorbent article in the form of an assembled T-shaped brief according to the present invention;

FIG. 8 is an isometric view of a third embodiment of a disposable absorbent article in the form of a ready-to-wear pull-up according to the present invention;

FIG. 9 is a top plan view of a second embodiment of a disposable absorbent article according to the present invention showing a diaper having two minihook fastener elements and a nonwoven;

FIG. 10 is an isometric view of a disposable absorbent article in the form of an assembled diaper according to the present invention;

FIG. 11 is a scanning electron microscope photograph showing a top plan view of a preferred fastener element according to the present invention;

FIG. 12 is a similar photograph showing a side elevational view thereof;

FIG. 13 is a fragmentary sectional view of a fastener element according to the present invention, to an enlarged scale, secured to a nonwoven as a tape tab; and

FIG. 14 is a fragmentary sectional view of a fastener element according to the present invention, to an enlarged scale, secured to a nonwoven as a patch or strip.

### DETAILED DESCRIPTION OF THE PREFERRED EMBODIMENTS

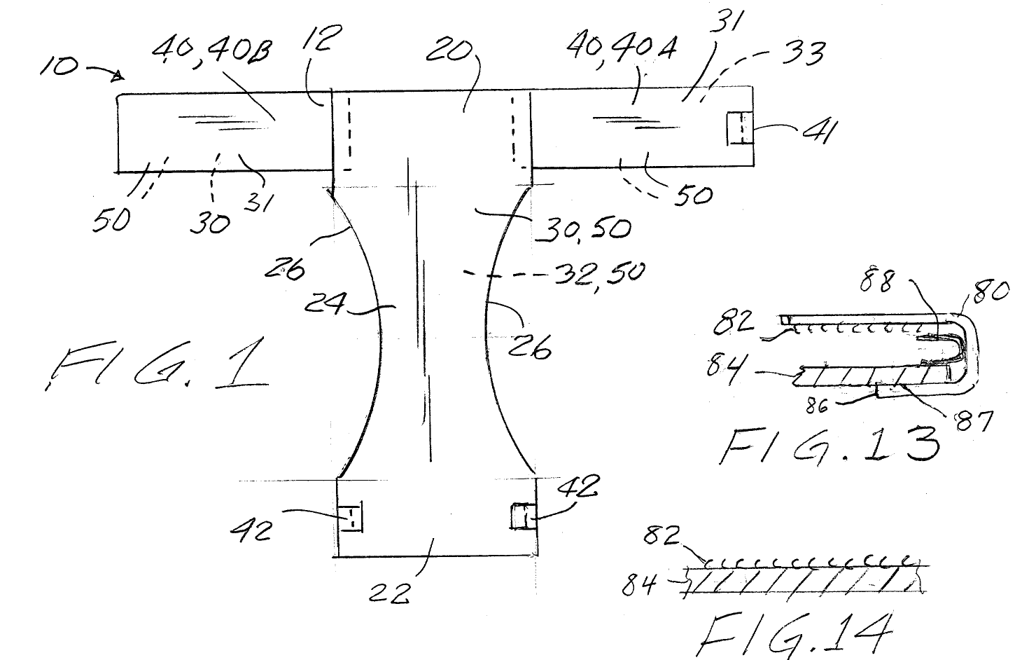

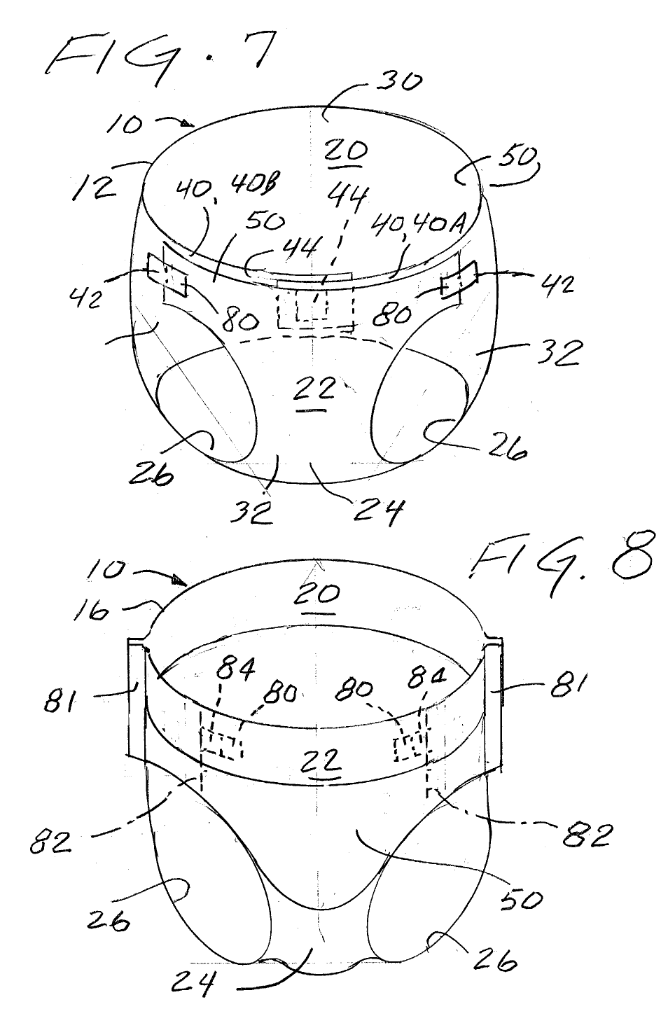

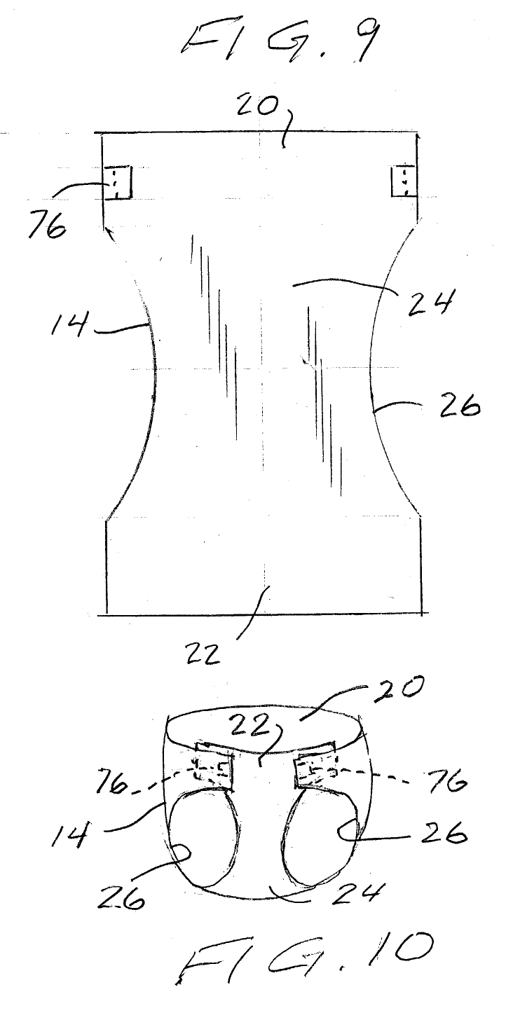

Referring now to the drawing, and in particular to FIGS. 1 and 7 thereof, therein illustrated is an absorbent article according to the present invention, generally designated by the reference numeral 10. The absorbent article 10 is in the form of a T-shaped brief 12, although it may alternatively be in the form of a diaper 14 (as illustrated in FIGS. 9 and 10) or a pull-up 16 (as illustrated in FIG. 8). Each article 10 has a back portion 20, a front portion 22 and a crotch portion 24 connecting the back and front portions 20, 22. As is conventional in such constructions, the crotch portion 24, either alone or in combination with one or both of the back and front portions 20, 22, defines leg openings 26 adapted to snugly receive the wearer's legs. The leg openings 26 may be elasticized for a better, more leakproof connection with the wearer's legs.

The construction typically includes a frontsheet 30 (also referred to as a body-facing sheet or a liner, cover or top sheet) and a backsheet 32 (also referred to as a garment-facing sheet or barrier sheet). An absorbent core (not shown) is disposed intermediate the frontsheet 30 and backsheet 32 and is typically formed of wood fluff or other absorbent material (and optionally contains gel-forming super-absorbent polymeric particles). Optionally an acquisition/distribution layer (not shown) is disposed intermediate the absorbent core and the frontsheet 30, or may even include as a part thereof the frontsheet 30. For expository purposes, the drawings herein illustrate only the frontsheet 30 and backsheet 32 of the constructions along with any fastener assemblies, thus ignoring for expository purposes the absorbent core, any internal acquisition/distribution layer, and the like.

Giving the T-shaped brief 12 its name, a pair of wings 40 extend laterally from respective lateral edges of the back portion 20. When the brief 12 is first placed on the wearer, the free ends of the two wings 40 are brought around the sides and front of the waist of the wearer and make a partially overlapping connection about the front of the waist of the wearer. According to the present invention, one of the wings 40 has at least one hook-type fastener element 4 body-facing surface 31 thereof, and the front portion 22 adjacent the top thereof (when the brief 12 is being worn) has at least a first laterally spaced pair of the hook-type fastener elements 42 on the frontsheet 30 thereof (the frontsheet 30 of the front portion 22 facing towards the wearer when the brief 12 is being worn). The fastener elements 41, 42 are exemplified herein as tape tab fastener elements permanently secured—in use in a non-overlapping relationship—to both the front and back (i.e., frontsheet 30 and backsheet 32) of an edge of a back portion 20 or front portion 22, or to both the front and back (i.e., a body-facing surface 31 and a garment-facing surface 33) of an edge of a wing 40, by an intermediate member such as a conventional tape tab 80. See FIG. 14 and commonly owned U.S. application Ser. No. 10/266,420, filed Oct. 8, 2002, which is hereby incorporated herein by reference.

Thus, as illustrated in FIG. 7, once the two wings 40 are placed in partially overlapping connection, they, in effect, form (with the back portion 20) a belt 44 encircling the waist of the wearer. The front portion 22 is then passed through the legs of the wearer and raised in front of the wearer until the first laterally spaced pair of fastener elements 42 secured thereto engage the exposed outer surface of the belt 44. Each wing 40 includes as at least the garment-facing surface 33 thereof (facing away from the wearer when the brief 12 is worn) the nonwoven 50.

The wings 40 may be elastic, non-elastic or partially elastic. Elastic wings may be made of a composite of an elastic film laminated between two nonwovens preferably spunbonds. A suitable composite is available from Treadgar Company under the designation FABRIFLEX 307. Even where the wings 40 are non-elastic, the degree of overlap in the connection between the wings 40 (to form the belt 44) will vary with the waist size of the wearer. While a large-waisted person may have each of the first pair of fastener elements 42 contacting a respective wing 40, a small-waisted person may have both of the pair of fastener elements contacting a single wing 40 which almost completely overlaps the other wing 40. In a conventional T-shaped brief this would present a problem in suitably locating the loop landing zones (that is, the conventional female fastening elements) along the garment-facing surface 33 each wing 40 if the brief were to be suitable for use by both large- and small-waisted persons.

On the other hand, when the T-shaped brief 12 is made according to the present invention so that the fastener elements 41, 42 do not require landing zones and may engage directly with any portion of the nonwoven surface constituting the frontsheet 30, backsheet 32 or wings 40 of the brief, the accommodation of both small and large-waisted persons by a single brief 12 with elastic wings 40 is substantially simplified. The advantage afforded by the present invention is especially noteworthy when the wings 40 are at least partially elastic since there is a tendency for a wearer (or caregiver who puts the brief on the wearer) to stretch one wing 40 more than the other wing 40 during the process of fastening the wings 40 to form the belt 44. This is less of a problem where the back portion 20 as well as the wings 40 are elastic, but more of a problem where the back portion 20 is not elastic and thus the stretching of the belt 44 is limited to the wings 40.

For the extra-large waisted wearer, the wings 40 need not be secured together at the front of the wearer to form (with the back portion 20) a belt 44. Instead, they are secured by the wearer to opposite lateral edges of the front portion 22 by fastener elements 42 so that the wings 40 form a belt 44 including both the front and back portions 22, 20.

The T-shaped brief 12 described above and illustrated in FIGS. 1 and 7 is susceptible of various improvements through relocation of the first pair of fastener elements on the front portion and/or the deployment of additional (auxiliary) fastener elements on either the front portion or the belt. These improvements are made possible by the ability of the fastener element to make an engagement without the presence of a landing zone.

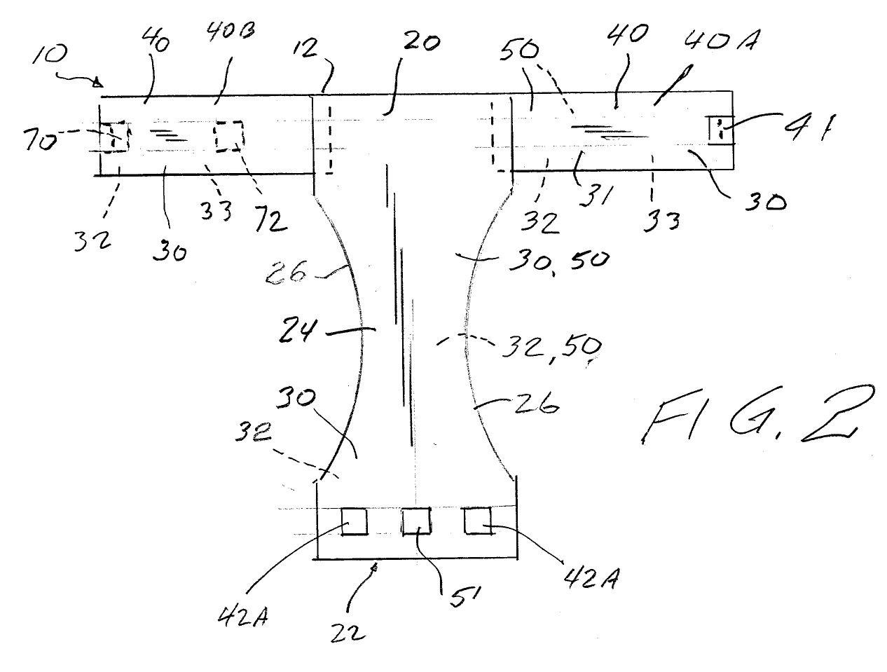

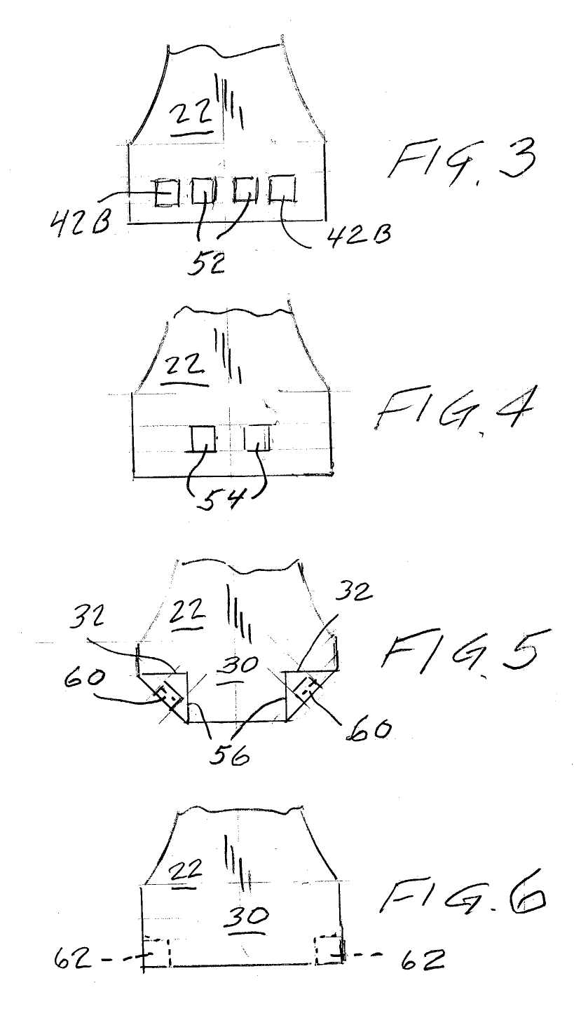

Referring now to FIG. 2, a single auxiliary fastener element 51 is positioned centrally between the first pair of fastener elements 42A (the latter being generally adjacent the lateral edges and top of the front portion 22). Thus, the tendency of the front portion 22 to billow forwardly from the belt 44 and form a pocket is overcome. The result is a flatter front portion 22 which more closely conforms to the contours of the belt 44 for a neater appearance and for reducing the possibility of spillage therebetween.

Referring now to FIG. 3, alternatively a second laterally spaced pair of fastener elements 52 are disposed between a first pair of fastener elements 42B and adjacent the top of the front portion 22, again acting to provide a flatter front portion 22.

Referring now to FIG. 4, alternatively the first pair 54 of fastener elements is simply substantially spaced inwardly from the lateral edges of the front portion 22 (e.g., by 1-2 inches) so as to make a connection with the belt 44 which is closer to the vertical centerline of the front portion 22 (and presumably the wearer), with the specific advantages noted hereinabove.

The fastener elements 42A, 42B, 51, 52 and 54 have been exemplified herein as patch or strip elements which are directly and permanently secured in back-to-face overlapping relationship to a frontsheet 30, backsheet 32 or wing 40 (e.g., by a hot melt or pressure-sensitive adhesive, ultrasonically, etc). See, for example, FIG. 13.

Whereas FIGS. 1-4 require the fastener elements 41, 42, 42A, 42B, 52, 54 to be disposed on the frontsheet 30 of the front portion 22 of the T-shaped brief 12 so as to make the desired contact with the garment-facing wing surface 33 or backsheet 32 of the belt 44, this is not always a practical arrangement. While the backsheet or garment-facing surface 32 of the front portion 22 is typically a spunbond nonwoven in order to provide strength and desirable feel to a caregiver, the body-facing frontsheet 30 of the T-shaped diaper 12 may be made of more fragile material selected for its properties as a liner or as part of an acquisition/distribution layer. In such instances, the frontsheet 30 may not provide a sufficiently secure base for the fastener elements.

Accordingly, in the variant illustrated in FIG. 5, the free end corners 56 formed by the top and an adjacent lateral edge of the front portion 22 are turned under and secured in that position (for example, by a few drops of hot-melt glue applied therebetween). The pair of tape tab fastener elements 58 are then attached on the turned-under corners 56, on the original backsheet 32 of the front portion 22 (now facing the wearer). Since it is preferred to manufacture the T-shaped brief with both the front and back portions 22, 20 having similar lateral (horizontal) extensions, this solution has the advantage of enabling such a preferred production technique while still positioning the fastener elements 58 inwardly of the lateral edges and on the original backsheet 32 of the front portion 22.

In the variant illustrated in FIG. 6, the pair of patch fastener elements 62 are secured to the backsheet 32 of the front portion 22 (adjacent the top thereof)—without any turning under of corners 56—and the top segment of the front portion 22 is then inserted (e.g., tucked) under the belt 44 so that the fastener elements 62 on the backsheet 32 of the front portion 22 will engage the frontsheet 30 of the belt 44 (actually the body-facing surface 31 of a wing 40). The ability of the fastener element of the present invention to engage any portion of the belt backsheet 32 (rather than a limited loops landing zone) enables this “blind” operation to proceed easily and rapidly even though the fastener elements 62 are not themselves visible during the tucking-in step. In this situation, the pressure of the belt 44 on the top of the front portion 22 assists in maintaining the top of the front portion 22 flat and in engagement with the frontsheet 30 of the belt 44. It will be appreciated that the fastener elements 62 may be disposed adjacent the lateral edges of the front portion 22 or more centrally thereof as the retention of the front portion 22 in its desired position relies not simply on the fastener elements 62, but also on the squeezing of the top of the front portion 22 between the wearer and the belt 44.

When a small-waisted person wears a conventional T-shaped brief, the first or outer wing 40A (with the primary fastener element) overlaps the second or inner wing 40B (without the primary fastener element) so that the primary fastener element secures the free end of the first wing 40A to the second wing 40B (albeit more to one side of the wearer than to the front of the waist of the wearer). This leaves the free end of the second wing 40B (beyond the fastening point) supported only by the pressure exerted thereon by the first wing 40A. Accordingly, the free end of the second wing 40B will often simply drop down and hang below the belt 44, thereby presenting a messy, undignified appearance. Further, the free end can poke out and forward to present an unsightly bulge in the outer pants of the wearer. Accordingly, referring now to FIG. 2, an auxiliary fastener element 70 is preferably provided on the outer garment-facing surface 33 of the second wing 40B at or adjacent the free end thereof. This auxiliary fastener element 70 may be used to fasten the free end of the second wing 40B to the overlapping inner body-facing surface 31 of the first wing 40A at a point remote from the free end of the first wing 40A, thereby to maintain the free end of the second or inner wing 40B neatly in place under the first or outer wing 40A.

When a small-waisted person wears a conventional T-shaped brief, the engagement of the first or outer wing 40A (with the primary fastener element) on the inner or second wing 40B (without the fastener element) occurs not at the center front of the waist of the wearer, but more to one side of the wearer. This can result in the formation of an outwardly protruding pocket at the front center of the briefs, intermediate the inner and outer wings 40B, 40A, thereby preventing a messy, undignified appearance and increasing the possibility of leakage through the pocket. Accordingly, still referring now to FIG. 2, an auxiliary hook fastener 72 is preferably provided on the outer garment-facing surface 33 of the second or inner wing 40B more adjacent the main segment of the back portion 20. Thus the auxiliary fastener element 72 would be located on the front center of the brief 12 when worn by a small-waisted person and provide another point of engagement between the two wings 40A, 40B, so as to prevent formation of a pocket therebetween.

It will be appreciated that the provision of an auxiliary fastener element 70, 72 on the outer garment-facing surface 33 of the second or inner wing 40B may serve two different functions depending on how far the auxiliary fastener element is spaced from the free end of the second or inner wing 40B. Thus in FIG. 2, the auxiliary fastener element 70 on the second or inner wing 40B is adjacent the free end thereof, thereby to maintain that free end under the first or outer wing 40A; while the auxiliary fastener element 72 is well spaced from the free end of the second or inner wing 40B, thereby to maintain the overlapping portions of the wings 40A, 40B in contact in front of the wearer and avoid formation of a pocket therebetween.

It will be appreciated by those skilled in the art that the engagement illustrated in FIG. 2 as provided by auxiliary fastener 72 may alternatively be provided at about the same location by an auxiliary fastener element (not shown) on the inner body-facing surface 31 of the first or outer wing 40A at a corresponding location.

As the auxiliary fastener elements 70 and 72 perform for the small-waisted wearer different functions (one 70 maintaining the free end of the inner wing under the outer wing, and one 72 maintaining an engagement of the inner and outer wings more-or-less in the front center of the waist of the wearer), both auxiliary fastener elements 70, 72 may be provided on a single T-shaped brief 12 so that either may be employed or both may be employed simultaneously. Indeed, many of the auxiliary fastener elements described hereinabove may profitably be employed together.

Referring now to FIGS. 9 and 10, it will be appreciated by those skilled in the art that the same or analogous improvements in the fit, appearance and economy of diapers 14 according to the present invention, with tape tab or patch fastener elements 76 (adjacent opposed lateral edges of the back portion 20), may be realized.

A pull-up 16 is characterized by at least one permanent side seam 81, preferably two heat-sealed side seams 81, but is often provided with a line of weakness 82, or preferably a pair of lines of weakness 82 (typically extending from each leg opening 26 to the top of the front waist portion 32), for use in an emergency situation requiring rapid removal of the pull-up from the wearer. For example, emergency circumstances may not permit the wearer to remove the trousers, slacks or shoes so that the easiest and fastest removal of the pull-up is achieved by tearing it along the lines of weakness (which may be the heat-sealed side seams), thereby to enable removal thereof in the same manner as a conventional diaper. However, to enable this pull-up to be replaced or another pull-up to be put on the wearer, again without removal of trousers, slacks or shoes, a tape tab fastener element according to the present invention 84 is provided along one side of each line of weakness 82 for potential engagement with a nonwoven 50 on the other side of each line of weakness. This enables replacement of the original pull-up or the putting on of a new pull-up much in the manner of a conventional diaper.

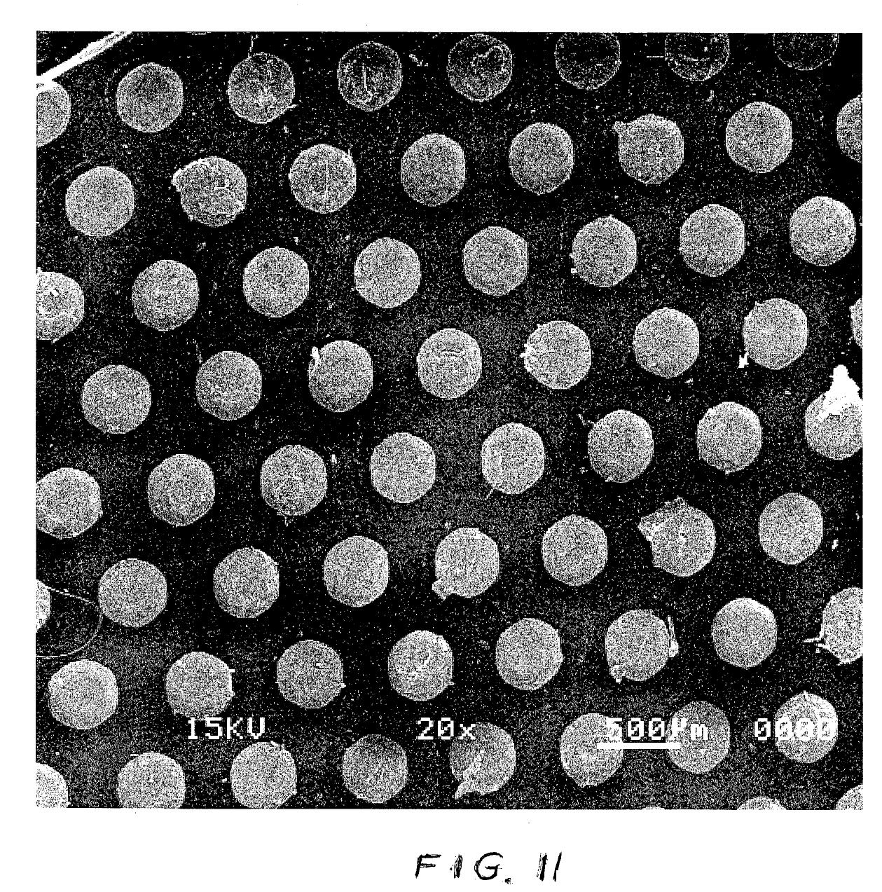

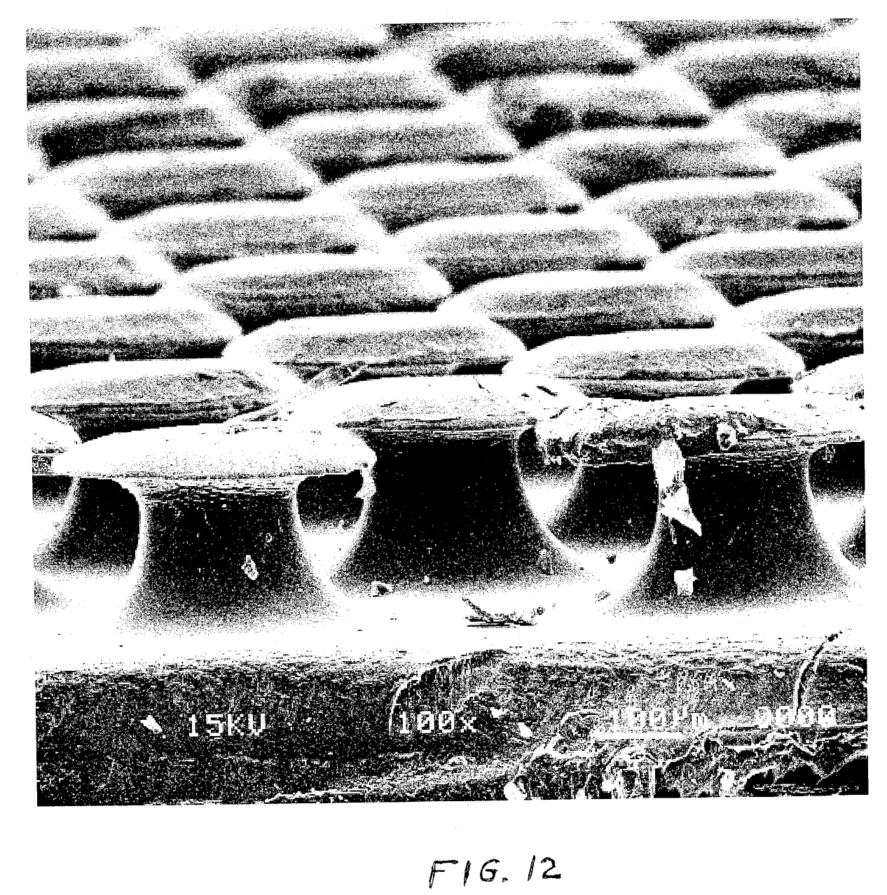

Referring now to the microphotographs of FIGS. 11 and 12, therein illustrated are preferred fastener elements 42 according to the present invention. As illustrated in FIG. 11, the hooks have a mushroom-like appearance, the substantial circularity of the mushroom caps facilitating a uniform and close spacing at the exposed outer surfaces of the caps. The fastener element 42 must have the hooks (also called “pins”) very closely spaced apart, an average density of at least 1600 pins per square inch (PPSI) preferably defining the exposed outer surface of the fastener element 42. The pins should be spaced, on average, less than about 300 microns (preferably less than about 275 microns) apart at the exposed outer surface thereof. To enable the pins to be so closely and regularly spaced, they are preferably substantially circular, at least at the exposed outer surface thereof. Preferably each pin has at a base thereof an average diameter of at least 300 (preferably 390) microns and at the free end thereof an average diameter of at least 375 (preferably 427) microns. Such a pin design allows the desired density or packing of at least 1600 PPSI to be achieved.

In order to achieve useful peel and tear strengths, the pin-bearing surface area of the fastener element (measured at the surface supporting the bases of the pins) should be at least 0.5 inch² Where the fastener element is of a rectangular design, preferably it is at least one inch in width by a half inch in length, optimally at least two inches by one inch. While the fastener elements according to the present invention have been illustrated as rectangular in outline, they may also be of other polygonal designs or even circular.

While the pins have been described in terms of their diameters at the base and at the free end thereof, where the pins are non-circular, the term “diameter” as used herein should be taken as indicating the minimum dimension (e.g., the minimum dimension at the base or at the free end of the pin).

A preferred fastener element is available from Binder Macroplast LP of Germany under the trade designation #42-288-HX-200-PP3.

Those skilled in the art will appreciate that not all of the fastener assemblies according to the present invention will require a peel strength of 60 grams per square inch and a shear strength of 1300 grams per square inch. For example, in FIG. 2, the auxiliary fastener element 51 according to the present invention on the front portion 22 and the two fastener elements according to the present invention on the garment-facing surface of wing 40B may have lower peel and shear strengths since they are not relied upon for maintenance of the T-shaped brief 12 in its basic essential orientation on the wearer.

The nonwoven 50, which defines an exposed outer surface of at least one of the frontsheet 30, backsheet 32, and wing 40, contains a plurality of bonded fibers. The fibers may be bonded by a variety of different means well-known in the art including thermal bonding, chemical bonding (e.g., by latex) hydroentanglement, and the like. Similarly, the fibers may be point bonded or area bonded, the area bonding typically indicating either a very large point or a plurality of very close points. Preferably the fibers are point bonded by a thermal technique such as calendering to produce a fuzzy nonwoven having a percentage bonding area of about 15-30%.

The bonded fibers define therebetween a plurality of interstices (also called pores, apertures, openings, etc.). A preferred nonwoven is a 13-50 gsm spunbond wherein the fibers have an average size of 1.3 to 3.0 denier and the average size of the interstices is at least 644 microns. While the bonding pattern may be uniform in both the machine direction (MD) and the cross direction (CD) of the nonwoven web, preferably the pattern differs for the MD and the CD. See, for example, U.S. Pat. No. 6,537,644 which discloses an orthogonally differentially bonded nonwoven.

It will be appreciated that the backsheet may be formed of the above-identified nonwoven with a polypropylene or polyethylene film backing or as a laminate or like composite of a plurality of nonwovens.

While the nonwoven is preferably a spunbond having a basis weight of 13-50 grams per square meter (gsm), slightly higher basis weights of 20-40 gsm (optimally 34 gsm) are preferred for use on the wings of a T-shaped brief, while relatively lower basis weights of 15-30 gsm (optimally 20 gsm) are preferred for the backsheet of the front and back portions.

A preferred non-elastic spunbond nonwoven is available from First Quality Nonwovens, Inc. under the trade designations “PILLOWBOND SPUNBOND” and “SPUNBOND NW147”.

Within the limits and ranges described hereinabove, the fastener element and nonwoven are selected so that the pins of the fastener element enter into the interstices of the nonwoven when the exposed outer surfaces of the fastener element and the nonwoven are mutually engaged.

The number of fibers and the extent to which they are bonded together in the nonwoven provide the nonwoven with the strength necessary to provide a high level of shear strength. The size of the spacing between adjacent surfaces of the pins of the fastener element at the exposed outer surface thereof is a factor of major significance providing the high level of shear strength. It is believed that the pins must have dimensions at the free ends thereof enabling the free ends to enter into the interstices (and possible through the interstices) defined by the fibers of the nonwoven. On the other hand, there is some evidence to the effect that the free ends of the pins need not pass through the interstices of the nonwoven in order to mechanically engage the fibers of the nonwoven.

The pore size in the nonwoven (that is, the size of the interstices formed by the fibers) typically affects the peel strength, a higher pore size providing greater peel strength for the fastener elements according to the present invention. While peel strength is in reality not of high importance as a practical matter for most applications, it often plays an importance role psychologically in providing reassurance against accidental opening of a fastened assembly. Accordingly, an elastic composite, as described hereinabove, is preferred for the wings 40 as the pore size will increase with stretching of the composite, thereby providing a good balance between peel strength and shear strength under actual use conditions (that is, when the composite is stretched by at least 100%). The pore size of the composite under actual use conditions may strongly affect the degree to which the free ends of the pins can enter into or even pass through the interstices of the outer surface of the nonwoven of the composite.

Contrary to expectations, the preferred fastener element of the present invention is preferred for use only with conventional nonwovens and has not been found to be particularly suitable for use with either the conventional VELCRO mini-loop fastener elements or the conventional loop-providing materials intended for use with conventional hook-type VELCRO fastener elements. When the preferred fastener element is used with a conventional nonwoven, especially the preferred nonwoven spunbond described hereinafter, the fastener assembly according to the present invention has a shear strength substantially higher (and frequently several times higher) than that obtained by a conventional VELCRO-like hook-type fastener element with a comparable conventional nonwoven.

The test followings procedures were used for determining the peel and shear resistance of a hook-type tape tab attached to a nonwoven. A 9.8 lb weight is placed on one end of a nonwoven samples. The sample is then stretched out until the weight starts to move. The hook-type tape sample (2 inches wide by 1 inch in length) is then applied to the nonwoven (the nonwoven being oriented in the same direction as it would be in actual use), and the rubber-covered hand-operated roller is rolled five times across the 2 inch width of the hook-type tape tab.

A constant rate of extension tensile tester (e.g., INSTRON Model 4442) is then set up with a full scale load of 10 lbs. and a cross-head speed of 12 inches per minute. For the tape peel test, the draw separation is set at 1 inch and the test length at 2 inches, while for the tape shear test, the draw separation is set at 4 inches and the test length at 4 inches.

In the tape peel test, the nonwoven is inserted in the lower clamp and its jaws then closed, and the hook-type tape tab is inserted in the upper clamp and its jaws then closed. In the tape shear test, the nonwoven ends are inserted into respective upper and lower clamps, and the jaws of each closed. Testing is then begun, with the type of failure being noted (either nonwoven or tape) and the maximum load and load at break being reported.

The specification herein of a “frontsheet” or “backsheet” of a wing 40 or belt 44 refers to the “wearer-facing surface” or “garment-facing surface,” respectively, of the wing 40 or belt 44.

The specification herein of an absorbent article as having or including a “fastener element” according to the present invention encompasses both a tape tab fastener element and a patch or strip fastener element except where the context of the specification requires otherwise.

The patch or strip fastener element is illustrated in FIG. 14 as a fastener element 82 directly and permanently secured in back-to-face overlapping relationship to a substrate 84, such as a frontsheet 30 or backsheet 32 of a rear portion 20 or front portion 22, or a body-facing wing surface 31 or garment-facing wing surface 33 of a wing 40. The fastener element 82 may be secured to the substrate 84 by a variety of conventional means including hot melt adhesive, pressure-sensitive adhesive, ultrasonic welding, etc.

The tape tab fastener element is illustrated in FIG. 13 as permanently secured (in non-overlapping relationship when in use) to a base 84 such as both the front and back (i.e., frontsheet 30 and backsheet 32) of an edge of a back portion 20 or front portion 22, or to both the front and back (i.e., body-facing surface 31 and garment-facing surface) of an edge of a wing 40, by an intermediate member such as a conventional tape tab 80. The tape tab 80 has disposed thereon at one end (the free or fastening end) a fastener element 82 and at the other end an edge of substrate 84. The tape tab 80 includes at the substrate end a main body 86 permanently secured at 87 to the front sheet 30 or backsheet 32 or to the body-facing surface 31 or garment-facing surface 33, and a short strip of paper 88 (such as a release paper) permanently secured at the substrate end to the other of the frontsheet 30 or backsheet 32 or the other of the body-facing surface 31 or garment-facing surface 33. The paper 88 strengthens the connection of the tape tab 80 to the substrate edge and at the same time provide a surface to which the fastener element will not engage. (It will be appreciated that such surface of the paper 88 may or may not also be one with which adhesives will not engage.) The fastener element portion is initially folded over the edge prior to deployment into actual use.

To summarize, the present invention provides an absorbent article which incorporates a fastener assembly consisting exclusively of a minihook fastener element, without any miniloop fastener element beyond a conventional nonwoven material, such as a spunbond nonwoven. The fastener assembly provides a peel strength of at least 60 grams per square inch and a shear strength of at least 1300 grams per square inch and provides size adjustability and/or other features at the cost of only additional minihook fastener elements. The disposable article may be a diaper, a T-shaped brief, or a pull-up.

Now that the preferred embodiments of the present invention have been shown and described in detail, various modifications and improvement thereon will become readily apparent to those skilled in the art. Accordingly, the spirit and scope of the present invention is to be construed broadly and limited only by the appended claims, and not by the foregoing specification.

1. A disposable absorbent article having a frontsheet and a backsheet, comprising:
(A) a nonwoven defining an exposed outer surface of at least one of a frontsheet and a backsheet, said nonwoven containing a plurality of bonded fibers and a plurality of interstices defined thereby; and
(B) a minihook fastener element forming a plurality of pins defining an exposed outer surface thereof;
said pins of said fastener element and said fibers and said interstices of said nonwoven being configured and dimensioned to provide, when said exposed outer surfaces of said fastener element and said nonwoven are mutually engaged, a peel strength of at least 60 grams per square inch and a shear strength of at least 1300 grams per square inch.

2. The article of claim 1 comprising a diaper having a back portion, a front portion and a crotch portion connecting said back and front portions, one of said back and front portions including a laterally spaced pair of said fastener elements and the other of said back and front portions including said nonwoven.

3. The article of claim 1 comprising a T-shaped brief having a back portion, a front portion, a crotch portion connecting said back and front portions, and a pair of wings extending laterally from respective lateral edges of said back portion for partially overlapping connection about the front of a waist of a wearer, one of said wings having at least one of said fastener elements, and said front portion having at least a first laterally spaced pair of said fastener elements and both of said wings having said nonwoven.

4. The article of claim 1 comprising a pull-up having a back portion, a front portion and a crotch portion connecting said back and front portions, said back and front portions being adapted to be secured together in an emergency situation exclusively by at least one of said fastener elements and said nonwoven.

5. The article of claim 3 wherein said front portion includes a single auxiliary fastener element between said first pair of fastener elements of said front portion.

6. The article of claim 3 wherein said front portion includes a second laterally spaced pair of said fastener elements, said second pair being disposed between said first pair.

7. The article of claim 3 wherein said first laterally spaced pair of fastener elements is substantially spaced inwardly from the lateral edges of said front portion.

8. The article of claim 3 wherein said lateral edges of said front portion adjacent a top thereof are secured in a turned under orientation to the frontsheet of said front portion, said first laterally spaced pair of fastener elements being on the backsheet of said turned under lateral edges.

9. The article of claim 3 wherein said first laterally spaced pair of said fastener elements is disposed on the frontsheet of said front portion, and said front portion is disposed over at least one of said wings.

10. The article of claim 3 wherein said first laterally spaced pair of said fastener elements is disposed on the backsheet of said front portion, and said front portion is partially disposed under at least one of said wings.

11. The article of claim 3 wherein the partially overlapped one of said wings has an auxiliary fastener element on the backsheet thereof substantially adjacent the free end thereof, thereby to enable fastening of said free end to the body-facing surface of the overlapping one of said wings.

12. The article of claim 3 wherein the partially overlapped one of said wings has an auxiliary fastener element on the backsheet thereof on an intermediate portion substantially spaced from the free end thereof, thereby to enable fastening of said intermediate portion to the body-facing surface of the overlapping one of said wings.

13. The article of claim 3 wherein said wings are at least partially elastic and include said nonwoven as an outer surface thereof.

14. The article of claim 1 wherein said nonwoven is a spunbond.

15. The article of claim 1 wherein said nonwoven is a backsheet.

16. The article of claim 1 wherein said nonwoven is 13-50 gsm, and said minihook fastener element has a density of at least 1600 PPSI and a pin-bearing surface area of at least 0.5 inch², said pins being substantially circular and spaced less than about 300 microns apart at an exposed outer surface thereof.

17. The article of claim 1 wherein said nonwoven is 13-50 gsm, said fibers at the exposed outer surface thereof have an average size of about 1.3 to 3.0 denier, and the average size of said interstices is at least 644 microns, and each said pin has a diameter
(i) at the base thereof of at least 300 microns, and
(ii) at the free end thereof of at least 375 microns.

18. A disposable absorbent article having a frontsheet and a backsheet, comprising:
(A) a nonwoven defining an exposed outer surface of at least one of a frontsheet and a backsheet, said nonwoven containing a plurality of bonded fibers and a plurality of interstices defined thereby; and
(B) a minihook fastener element having
(i) an average density of at least 1600 pins per square inch (PPSI) defining an exposed outer surface thereof, said pins being substantially circular and on average spaced less than about 300 microns apart at an exposed outer surface thereof, and
(ii) a pin-bearing surface area of at least 0.5 inch².

19. The article of claim 18 wherein said pins are on average spaced less than about 275 microns apart at an exposed outer surface thereof.

20. The article of claim 18 wherein said nonwoven has a basis weight of 13-50 gsm.

21. The article of claim 18 comprising a diaper having a back portion, a front portion and a crotch portion connecting said back and front portions, one of said back and front portions including a laterally spaced pair of said fastener elements and the other of said back and front portions including said nonwoven.

22. The article of claim 18 comprising a T-shaped brief having a back portion, a front portion, a crotch portion connecting said back and front portions, and a pair of wings extending laterally from respective lateral edges of said back portion for partially overlapping connection about the front of a waist of a wearer, one of said wings having at least one of said fastener elements, and said front portion having at least a first laterally spaced pair of said fastener elements and both of said wings having said nonwoven.

23. The article of claim 18 comprising a pull-up having a back portion, a front portion and a crotch portion connecting said back and front portions, said back and front portions being adapted to be secured together in an emergency situation exclusively by at least one of said fastener elements and said nonwoven.

24. The article of claim 22 wherein said front portion includes a single auxiliary fastener element between said first pair of fastener elements of said front portion.

25. The article of claim 22 wherein said front portion includes a second laterally spaced pair of said fastener elements, said second pair being disposed between said first pair.

26. The article of claim 22 wherein said first laterally spaced pair of fastener elements is substantially spaced inwardly from the lateral edges of said front portion.

27. The article of claim 22 wherein said lateral edges of said front portion adjacent a top thereof are secured in a turned under orientation to the frontsheet of said front portion, said first laterally spaced pair of fastener elements being on the backsheet of said turned under lateral edges.

28. The article of claim 22 wherein said first laterally spaced pair of said fastener elements is disposed on the frontsheet of said front portion, and said front portion is disposed over at least one of said wings.

29. The article of claim 22 wherein said first laterally spaced pair of said fastener elements is disposed on the backsheet of said front portion, and said front portion is partially disposed under at least one of said wings.

30. The article of claim 22 wherein the partially overlapped one of said wings has an auxiliary fastener element on the backsheet thereof substantially adjacent the free end thereof, thereby to enable fastening of said free end to the body-facing surface of the overlapping one of said wings.

31. The article of claim 22 wherein the partially overlapped one of said wings has an auxiliary fastener element on the backsheet thereof on an intermediate portion substantially spaced from the free end thereof, thereby to enable fastening of said intermediate portion to the body-facing surface of the overlapping one of said wings.

32. The article of claim 22 wherein said wings are at least partially elastic and include said nonwoven as an outer surface thereof.

33. The article of claim 22 wherein said nonwoven is a spunbond.

34. The article of claim 22 wherein said nonwoven is the backsheet.

35. The article of claim 18 wherein said nonwoven is 13-50 gsm, said fibers at the exposed outer surface thereof have an average size of about 1.3 to 3 denier, and the average size of said interstices is at least 644 microns, and each said pin has a diameter
(i) at the base thereof of at least 300 microns, and
(ii) at the free end thereof of at least 375 microns.

36. The article of claim 18 wherein said pins of said fastener element and said fibers and said interstices of said nonwoven are configured and dimensioned to provide, when said exposed surfaces of said fastener element and said nonwoven are mutually engaged, a peel strength of at least 60 grams per square inch and a shear strength of at least 1300 grams per square inch.

37. A disposable absorbent article having a frontsheet and a backsheet, comprising:
(A) a nonwoven defining an exposed outer surface of at least one of a frontsheet and a backsheet, said nonwoven containing a plurality of bonded fibers and a plurality of interstices defined thereby; said nonwoven being a 13-50 gsm spunbond, said fibers having an average size of about 1.3 to 3.0 denier, and the average size of said interstices being at least 644 microns; and
(B) a minihook fastener element forming a plurality of pins defining an exposed outer surface thereof, said pins having an average diameter
(i) at the base thereof of at least 300 microns, and
(ii) at the free end thereof of at least 375 microns;
said pins of said fastener element entering into said interstices of said nonwoven when said exposed outer surfaces of said fastener element and said nonwoven are mutually engaged.

38. The article of claim 37 wherein said fibers are differentially bonded.

39. The article of claim 37 wherein said fibers are differentially point bonded.

40. The article of claim 37 comprising a diaper having a back portion, a front portion and a crotch portion connecting said back and front portions, one of said back and front portions including a laterally spaced pair of said fastener elements and the other of said back and front portions including said nonwoven.

41. The article of claim 37 comprising a T-shaped brief having a back portion, a front portion, a crotch portion connecting said back and front portions, and a pair of wings extending laterally from respective lateral edges of said back portion for partially overlapping connection about the front of a waist of a wearer, one of said wings having at least one of said fastener elements, and said front portion having at least a first laterally spaced pair of said fastener elements and both of said wings having said nonwoven.

42. The article of claim 39 comprising a pull-up having a back portion, a front portion and a crotch portion connecting said back and front portions, said back and front portions being adapted to be secured together in an emergency situation exclusively by at least one of said fastener elements and said nonwoven.

43. The article of claim 41 wherein said front portion includes a single auxiliary fastener element between said first pair of fastener elements of said front portion.

44. The article of claim 41 wherein said front portion includes a second laterally spaced pair of said fastener elements, said second pair being disposed between said first pair.

45. The article of claim 41 wherein said first laterally spaced pair of fastener elements is substantially spaced inwardly from the lateral edges of said front portion.

46. The article of claim 41 wherein said lateral edges of said front portion adjacent a top thereof are secured in a turned under orientation to the frontsheet of said front portion, said first laterally spaced pair of fastener elements being on the backsheet of said turned under lateral edges.

47. The article of claim 41 wherein said first laterally spaced pair of said fastener elements is disposed on the frontsheet of said front portion, and said front portion is disposed over at least one of said wings.

48. The article of claim 41 wherein said first laterally spaced pair of said fastener elements is disposed on the backsheet of said front portion, and said front portion is partially disposed under at least one of said wings.

49. The article of claim 41 wherein the partially overlapped one of said wings has an auxiliary fastener element on the backsheet thereof substantially adjacent the free end thereof, thereby to enable fastening of said free end to the body-facing surface of the overlapping one of said wings.

50. The article of claim 41 wherein the partially overlapped one of said wings has an auxiliary fastener element on the backsheet thereof on an intermediate portion substantially spaced from the free end thereof, thereby to enable fastening of said intermediate portion to the body-facing surface of the overlapping one of said wings.

51. The article of claim 41 wherein said wings are at least partially elastic and include said nonwoven as an outer surface thereof.

52. The article of claim 37 wherein said nonwoven is a spunbond.

53. The article of claim 37 wherein said nonwoven is the backsheet.

54. The article of claim 37 wherein said nonwoven is 13-50 gsm, and said minihook fastener element has a density of at least 1600 PPSI and a pin-bearing surface area of at least 0.5 inch², said pins being substantially circular and spaced less than about 300 microns apart at an exposed outer surface thereof.

55. The article of claim 37 wherein said pins of said fastener element and said fibers and said interstices of said nonwoven being configured and dimensioned to provide, when said exposed surfaces of said fastener element and said nonwoven are mutually engaged, a peel strength of at least 60 grams per square inch and a shear strength of at least 1300 grams per square inch.

56. A disposable absorbent article having a frontsheet and a backsheet, comprising:
(A) a nonwoven defining an exposed outer surface of at least one of a frontsheet and a backsheet, said nonwoven containing a plurality of bonded fibers and a plurality of interstices defined thereby; said nonwoven being a 13-50 gsm spunbond, said fibers having an average size of about 1.3 to 3.0 denier, and the average size of said interstices being at least 644 microns; and
(B) a minihook fastener element having
(i) an average density of at least 1600 pins per square inch (PPSI) defining an exposed outer surface thereof, said pins being substantially circular and on average spaced less than about 300 microns apart at an exposed outer surface thereof, and
(ii) a pin-bearing surface area of at least 0.5 inch²;
said fastener element and said nonwoven, when the exposed outer surfaces thereof are mutually engaged, having a peel strength of at least 60 grams per square inch and a shear strength of at least 1300 grams per square inch.
